(19) United States
(12) Patent Application Publication
(10) Pub. No.: US 2025/0001212 A1
(43) Pub. Date: Jan. 2, 2025

(54) RADIOTHERAPY APPARATUS, RADIATION DELIVERY METHOD, AND COMPUTER-READABLE STORAGE MEDIUM

(71) Applicant: Shanghai United Imaging Healthcare Co., Ltd., Shanghai (CN)

(72) Inventors: Liuyuan Zhou, Shanghai (CN); Shoubo He, Shanghai (CN); Peng Wang, Shanghai (CN); Gang Pan, Shanghai (CN); Cheng Ni, Shanghai (CN)

(21) Appl. No.: 18/760,132

(22) Filed: Jul. 1, 2024

(30) Foreign Application Priority Data
Jun. 30, 2023 (CN) 202310800371.7

(51) Int. Cl. A61N 5/10

(52) U.S. Cl. CPC A61N 5/1077; A61N 5/1045; A61N 2005/1089; A61N 2005/1094

(57) ABSTRACT

A radiotherapy apparatus includes a shielding cabin and a radiation delivery device. The shielding cabin is configured to shield radiation and has a treatment site disposed therein. The radiation delivery device is movably housed within the shielding cabin, and configured to generate treatment radiation and direct the treatment radiation to a target position at the treatment site.

### CROSS-REFERENCE TO RELATED APPLICATIONS

The present application claims the priority to Chinese Patent Application No. 202310800371.7, filed on Jun. 30, 2023, which is incorporated by reference herein in its entirety.

### TECHNICAL FIELD

The present disclosure relates to the field of radiation delivery devices, particularly to a radiotherapy apparatus, a radiation delivery method, and a computer-readable storage medium.

### BACKGROUND

Radiotherapy, also known as radiation therapy, is a treatment for malignant tumors or benign diseases using one or more types of ionizing radiation.

Existing radiotherapy solutions typically use a linear accelerator to generate the required beams and deliver them through a rotatable gantry to achieve multi-angle irradiation of tumors. Although the solution can achieve irradiation of tumors at different angles, the rotatable gantry is not only large in size, high in construction and maintenance costs, but also requires special treatment rooms with radiation shielding effect, which will further increase costs.

### SUMMARY

The first aspect of the present disclosure provides a radiotherapy apparatus. The radiotherapy apparatus includes a shielding cabin and a radiation delivery device. The shielding cabin is configured to shield radiation and has a treatment site inside. The radiation delivery device is movably housed within the shielding cabin and configured to generate treatment radiation and direct the treatment radiation to a target position at the treatment site.

In some embodiments, the radiotherapy apparatus further includes a guide rail arranged within the shielding cabin and configured to be movable along a length direction of the treatment site. The radiation delivery device is connected to the guide rail.

In some embodiments, the radiotherapy apparatus further includes a guide rail arranged around the treatment site. The radiation delivery device is connected to the guide rail.

In some embodiments, the radiation delivery device is configured to be movable relative to the guide rail.

In some embodiments, the radiation delivery device is configured to be fixed relative to the guide rail and move along with the guide rail.

In some embodiments, the radiation delivery device is configured to perform a spiral motion around the treatment site.

In some embodiments, the radiation delivery device includes a casing and a radiation generation system. The radiation generation system is disposed within the casing and configured to generate and emit treatment radiation. The casing is mounted to the guide rail, and the radiation generation system is configured such that an emission angle of the treatment radiation is adjustable.

In some embodiments, the radiation generation system includes an electron beam generator configured to generate electron beams, an electron beam accelerator configured to accelerate the electron beams derived from the electron beam generator, a target component configured to generate the treatment radiation using the accelerated electron beams, and a collimation device configured to adjust a beam shape of the treatment radiation.

In some embodiments, the shielding cabin includes a shielding cabin chamber and a shielding cabin door. The shielding cabin door is operably mounted to the shielding cabin chamber. The shielding cabin door and the shielding cabin chamber jointly enclose and form an accommodating space, and the treatment site is located inside the accommodating space.

In some embodiments, the radiotherapy apparatus further includes an imaging device configured to acquire an image of a region of interest at the target position.

In some embodiments, the radiotherapy apparatus further includes a control device communicatively connected to the imaging device and the radiation delivery device, and configured to control a position and/or an orientation of the radiation delivery device based on the image acquired by the imaging device.

In some embodiments, the imaging device is positioned orthogonally to the radiation delivery device relative to the target position.

In some embodiments, the imaging device is configured such that a position of the imaging device is adjustable relative to the treatment site synchronously with the radiation delivery device.

In some embodiments, the radiation delivery device is configured to be rotatable within a spherical angle range.

In some embodiments, the shielding cabin is configured to be rotated to allow for a supine or standing posture of an object.

The second aspect of the present disclosure provides a radiation delivery method based on a radiotherapy apparatus. The radiotherapy apparatus includes a shielding cabin and a radiation delivery device movably housed within the shielding cabin. The method includes delivering treatment radiation, by the radiation delivery device, to a target position at a treatment site disposed in the shielding cabin. The shielding cabin is configured to shield radiation.

In some embodiments, the radiotherapy apparatus includes a guide rail arranged within the shielding cabin and around the treatment site, and configured to be movable along a length direction of the treatment site. The radiation delivery device is connected to the guide rail and configured to be movable along the guide rail.

In some embodiments, the shielding cabin includes a shielding cabin chamber and a shielding cabin door. The shielding cabin door is operably mounted to the shielding cabin chamber. The shielding cabin door and the shielding cabin chamber jointly enclose and form an accommodating space. The treatment site is located inside the accommodating space.

In some embodiments, the radiotherapy apparatus further includes an imaging device and a control device communicatively connected to the imaging device and the radiation delivery device. The imaging device is configured to acquire an image of a region of interest at the target position. The control device is configured to control a position and/or an orientation of the radiation delivery device based on the image acquired by the imaging device.

The third aspect of the present disclosure provides a non-transitory computer-readable storage medium storing instructions. The instructions, when executed by a processor, cause the processor to perform the radiation delivery method according to the various embodiments described above.

Details of one or more embodiments of the present disclosure are presented in the accompanying drawings and description below. Other features, purposes and advantages of the present disclosure will become apparent from the specification, the accompanying drawings, and the claims.

### BRIEF DESCRIPTION OF THE DRAWINGS

In order to clarify the technical solutions in the embodiments of the present disclosure or conventional technical solutions, a brief introduction will be made to the drawings required in the descriptions of the embodiments or conventional techniques. It is obvious that the drawings described below are only some embodiments of the present disclosure. Those skilled in the art can obtain other drawings based on these drawings without creative efforts.

FIG. 1 is a schematic diagram of a structure of a radiotherapy apparatus according to an embodiment of the present disclosure.

FIG. 2 is a schematic diagram of the structure of the radiotherapy apparatus, viewing from another perspective, according to an embodiment of the present disclosure.

FIG. 3 is a schematic diagram of a structure of a radiation delivery device according to an embodiment of the present disclosure.

### DETAILED DESCRIPTION

The technical solutions in the embodiments of the present disclosure will be clearly and completely described in conjunction with the accompanying drawings. It is apparent that the described embodiments are only a part of the embodiments of the present disclosure, not including all embodiments. Based on the embodiments of the present disclosure, all other embodiments that can be obtained by those skilled in the art without creative efforts are within the protection scope of the present disclosure.

It should be noted that when a component is described as being “arranged on” another component, it may be directly arranged on the other component or there may exist an intermediate component. When a component is considered to be “arranged in” another component, it can be directly arranged in the other component or there may exist an intermediate component. When a component is considered to be “fixed to” another component, it can be directly fixed to the other component or there may exist an intermediate component.

Unless otherwise defined, all technical and scientific terms used in the present disclosure have the same meaning as understood by those skilled in the art of the technical field to which the present disclosure belongs. The terms used in the description of the present disclosure are intended to describe specific embodiments for illustrative purposes and are not intended to limit the disclosure. The term “and/or” as used in the present disclosure includes any and all combinations of one or more of the related described items.

The radiotherapy apparatus 100, which is to be protected by the present disclosure, can be configured to generate and deliver treatment radiation that can be applied to various treatments such as radiotherapy, radiographic examination, radiation testing, magnetic resonance imaging (MRI), digital radiography (DR), or computed tomography (CT). For example, treatment radiation may include photon beams (X-rays, y-rays, etc.), electron beams (low-energy electron beams, medium-energy electron beams, high-energy electron beams, etc.), fast neutron beams, proton beams, negative pion beams, other heavy particle beams, and so on. In addition, although a radiotherapy apparatus is exemplarily described in the present disclosure, it can be understood that the device can be used not only for radiotherapy-related treatments but also for various other types of treatments, such as radiation inspection of components, radiation processing of products, etc. Therefore, the term “radiotherapy apparatus” is not intended to limit the functionality and applications of the device.

The radiotherapy apparatus 100 can be, for example, placed in a treatment room, so that only the space inside the radiotherapy apparatus 100 is exposed to radiation, while other areas in the treatment room are not exposed to radiation. Therefore, there is no need to provide shielding effect for the entire treatment room. Optionally, the position of the radiotherapy apparatus 100 in the treatment room can be adjusted as needed. The size of the radiotherapy apparatus 100 may be, for example, from 2 to 4 meters in length, and 1.5-3 meters in width and height.

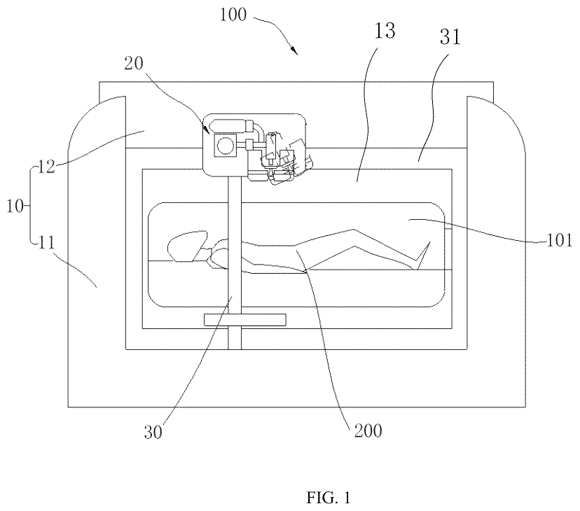

As shown in FIG. 1, in an embodiment of the present disclosure, the radiotherapy apparatus 100 includes a shielding cabin 10 and a radiation delivery device 20. The shielding cabin 10 has a treatment site 101 disposed therein where an object 200 to be examined or treated can be placed for treatment or examination. The treatment site 101 may include, for example, the position where a platform for carrying the object 200 is located. The shielding cabin 10 is configured to shield radiation. As a non-limiting example, the radiation that the shielding cabin 10 can shield includes one or more type of treatment radiation generated by the radiation delivery device 20, or ionizing or non-ionizing radiation generated by internal devices (e.g., the radiation delivery device 20) within the radiotherapy apparatus 100 or external devices (e.g., radiation sources outside the radiotherapy apparatus 100). The radiation delivery device 20 can be movably housed within the shielding cabin 10. When the radiation delivery device 20 is operating, it can generate treatment radiation and direct the treatment radiation to a target position on the object 200 at the treatment site 101. The radiation delivery device 20 can be adjusted in position relative to the treatment site 101, such that the treatment radiation can be directed into the target position at different angles.

It can be understood that by receiving the radiation delivery device 20 that generates radiation within the shielding cabin 10, the radiation can be shielded by the shielding cabin 10. For example, the shielding cabin 10 can shield the radiation generated inside the shielding cabin 10 from the outside. Alternatively or additionally, the shielding cabin 10 can shield the radiation generated outside the shielding cabin 10 from the treatment site 101 inside the shielding cabin 10. It is therefore, for example, able to avoid providing radiation shielding for the entire treatment room where the radiotherapy apparatus 100 is placed, thereby reducing costs. In addition, by adjusting the position of the radiation delivery device 20 relative to the shielding cabin 10, radiation can be delivered to the target position at different angles, which reduces the volume required to meet the requirements for multi-angle irradiation of the treatment radiation, thereby reducing costs, and also reduces the distance between the radiation delivery device 20 and the target position, further improving the therapeutic effect of the radiotherapy apparatus 100 during the radiotherapy process.

It should be noted that the target position refers to the location where the irradiation or delivery is expected to take place. As a non-limiting example, the target position can be an isocenter position, a specific volume around the isocenter, or multiple discrete point locations. As a specific example, the target position may include a region of interest (ROI), such as the location of a tumor or other lesions in the body of the object 200 where the radiotherapy apparatus 100 operates. That's to say, the radiotherapy apparatus 100 can accurately irradiate the tumor or lesion and achieve the therapeutic purpose. In addition, the target position may also be the location of various other living or non-living organisms. Exemplarily, the target position may be a position on irradiated experimental objects (e.g., dose verification phantoms, experimental animals, dose meters). As a non-limiting example, photon beams can be directed to the target position, allowing the photon beams to reach the target position and thus perform radiotherapy, examination, or testing on the object or target.

As can be seen from the above, the shielding cabin 10 can be configured to accommodate the object 200 and allow the object 200 to undergo irradiation in the shielding cabin 10. In operation, an installation position of the radiotherapy apparatus 100 can be changed according to the radiotherapy needs of the object 200, so that the object 200 can be accommodated in the shielding cabin 10 in a supine or standing posture to improve the comfort of the object 200 at the treatment site 101. Alternatively, the shielding cabin 10 can be rotated as a whole to allow for, for example, a supine or standing posture of the object 200. For example, the shielding cabin 10 can be rotated by a rotation mechanism in the treatment room. The object 200 can be any living or non-living body. For example, the object 200 can be a patient in need of radiotherapy, a phantom for dose verification, an animal for radiotherapy experiments, and so on. As a non-limiting example, the length of treatment site 101 can be not larger than 5 meters, not larger than 6 meters, between 2.2 meters and 5 meters, between 2.5 meters and 3 meters, or between 2 meters and 3 meters, and so on. As a non-limiting example, the cross-sectional site of the treatment site 101 can be not larger than 3 square meters, not larger than 10 square meters, not larger than 12 square meters, between 0.8 square meters and 2 square meters, between 0.5 square meters and 3 square meters, between 0.9 and 1.5 square meters, between 1 square meter and 3 square meters, or between 1.2 square meters and 2.9 square meters, and so on. For example, the smaller the size of the treatment site 101, the smaller the space occupied by the shielding cabin 10 can be, but the stricter the requirements for the size of the object 200, and the lower the comfort for the object 200 when it is a patient. Therefore, the size of the treatment site 101 can be set according to actual requirements or needs.

As shown in FIG. 1, in some embodiments, the shielding cabin 10 may include a shielding cabin chamber 11 and a shielding cabin door 12. The shielding cabin door 12 can be operably mounted to the shielding cabin chamber 11, and the shielding cabin door 12 and the shielding cabin chamber 11 jointly form and enclose an accommodating space 13. As such, the object 200 can enter the shielding cabin chamber 11 after the shielding cabin door 12 is opened, and then the shielding cabin door 12 can be automatically or manually closed on the shielding cabin chamber 11, thereby realizing the entrance of the object 200 into the accommodating space. It should be noted that the automatic opening and closing of the shielding cabin door 12 on the shielding cabin chamber 11 can be achieved under the control of an automated control system. The shielding cabin door 12 can also be set independently of the shielding cabin chamber 11, and the opening and closing of the shielding cabin door 12 with respect to the shielding cabin chamber 11 can be achieved using an external mechanical hand to grab and release the shielding cabin door 12. It can be understood that, for example, the shielding cabin chamber 11 and the shielding cabin door 12 of the shielding cabin 10 can be made of materials with a higher atomic number (e.g., lead, tungsten, cobalt, or their alloys) to provide self-shielding capabilities of the shielding cabin 10. However, not limited to the above manner, the shielding cabin chamber 11 and the shielding cabin door 12 of the shielding cabin 10 can be made of any other materials that provide shielding capabilities, such as composite materials, graphite, glass, rubber, plastic, etc.

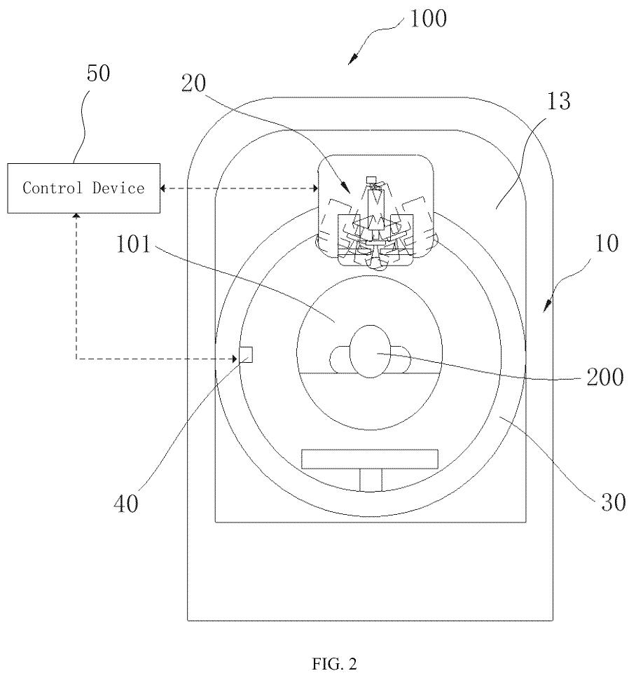

As shown in FIGS. 1 and 2, in some embodiments, the radiotherapy apparatus 100 includes a guide rail 30. The guide rail 30 can be arranged within the accommodating space 13, for example, by placing it within the shielding cabin chamber 11. The guide rail 30 can be arranged around the treatment site 101. The guide rail 30 is configured to be movable along the length direction of the shielding cabin 10 relative to the treatment site 101. For example, a circular bracket 31 is further provided and arranged on the inner walls of the radiotherapy apparatus 100 and around the accommodating space 13, and the guide rail 30 can be slidably mounted to the bracket 31 and engage with a track on the bracket 31, which thereby enables the guide rail 30 to be movable along the bracket 31, e.g., along the length direction of the shielding cabin 10. Optionally, the bracket 31 can be implemented as another guide rail orthogonal to the guide rail 30. The bracket 31 may also serve to connect and support the bed where the object 200 is placed, for example, at two ends of the bed.

In addition, for example, the radiation delivery device 20 can move around the treatment site 101 along the guide rail 30. It should be noted that the expression “moving along the guide rail 30” may include not only the case where the radiation delivery device 20 moves relative to the guide rail 30, but also the case where the radiation delivery device 20 is fixed relative to the guide rail 30 and moves along with the guide rail 30. As an example, the radiation delivery device 20 may be movably connected to the guide rail 30 and can move around the object 200 along the guide rail 30 (e.g., performing a circular or spiral motion). As another example, the radiation delivery device 20 may be fixedly connected to the guide rail 30, and the guide rail 30 can move around the treatment site 101 (e.g., performing a circular or spiral motion) and thus drive the radiation delivery device 20 to move around the treatment site 101. The radiation delivery device 20 is mounted within the shielding cabin 10 through the guide rail 30, and can move along the length direction of the treatment site 101 and/or around the treatment site 101, thereby changing the position of the radiation delivery device 20 relative to the object 200. FIGS. 1 and 2 show an exemplary structure of the guide rail 30, in which the guide rail 30 is a circular ring, however, the guide rail 30 can also be configured to be hexagonal, square, or rhombic. How the guide rail 30 moves and how the radiation delivery device 20 performs the circular motion on the guide rail 30 can be realized by those skilled in the art, and will not be further elaborated here.

Exemplary, the radiation delivery device 20 may perform only a circular motion around the object 200 along the guide rail 30 for a target object at the target position on the object 200 (e.g., a tumor tissue of a patient, an interested tissue or organ inside an animal body, or other target objects), perform only a linear motion along the body direction of the object 200, or perform a spiral motion combining the above two motions, until the radiation delivered by the radiation delivery device 20 during operation enter the target object at different angles and radiotherapy for the target object is realized. Alternatively, the radiation delivery device 20 may perform a circular motion around the object 200 by moving along the guide rail 30 or moving along with the guide rail 30, while the bed where the object 200 is placed moves in an orthogonal direction (e.g., the length direction of the bed) with respect to the guide rail 30, such that a spiral motion of the radiation delivery device 20 with respect to the object 200 can also be achieved.

Additionally, in some embodiments, the radiation delivery device 20 can rotate within a spherical angle range to achieve multi-angle irradiation treatment. For example, the radiation delivery device 20 is equipped with a mechanism that allows for rotation thereof. This mechanism typically involves, for example, motor-driven rotation or articulated joints, enabling precise movement within a defined spherical range. The radiation delivery device 20 may be further equipped with a precise angle control system, which ensures that the radiation delivery device 20 can be accurately positioned at various angles on the spherical surface. Control may be automated through programming or manually adjusted as needed.

It is to be noted that although FIGS. 1 and 2 show the guide rail 30 being arranged within the accommodating space 13, the guide rail can also be arranged outside the accommodating space 13 or on the side wall of the accommodating space 13, e.g., on the shielding cabin chamber 11 and/or the shielding cabin door 12. In these cases, the guide rail 30 can also move around the object 200 or the target position, and alternatively or additionally, it can move along the length direction of the treatment site 101.

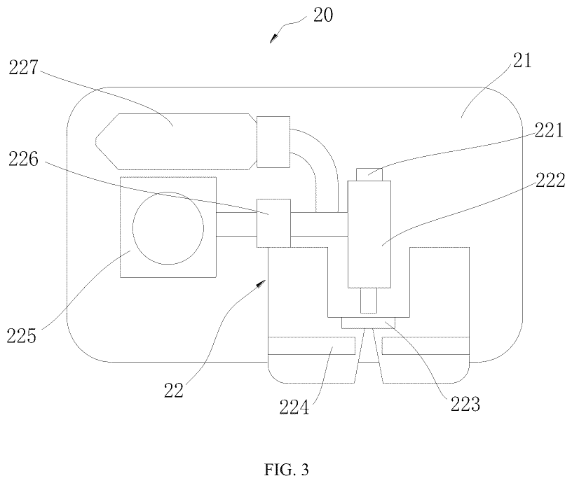

As shown in FIG. 3, in some embodiments, the radiation delivery device 20 includes a casing 21 and a radiation generation system 22. The radiation generation system 22 can be disposed within the casing 21 and configured to generate and emit treatment radiation. The casing 21 can be mounted to the guide rail 30, and the radiation generation system 22 can adjust the emission angle of the treatment radiation. As another example, the casing 21 is fixedly mounted to the guide rail 30, and a part or all of the radiation generation system 22 can swing to adjust the emission angle of the treatment radiation. In other words, the angle at which the treatment radiation is emitted during the operation of the radiation delivery device 20 can be adaptively adjusted according to the requirements of radiotherapy, thereby achieving comprehensive irradiation of the target position. It should be noted that the swinging angle and direction can be freely set as needed, without limitation thereto.

In some embodiments, in the case where the radiation delivery device 20 delivers photon beams, for example, the radiation generation system 22 may include an electron beam generator 221, an electron beam accelerator 222, a target component 223, and a collimation device 224. The electron beam generator 221 is configured to generate electron beams. The electron beam accelerator 222 is configured to receive and accelerate the electron beams derived from the electron beam generator 221. The target component 223 is configured to generate radiation (e.g., bremsstrahlung radiation or other types of radiation) using the accelerated electron beam from the electron beam accelerator 222 to form photon beams. The collimation device 224 is configured to adjust the beam shape of the photon beams from the target component 223. For example, the collimation device 224 may shape the photon beams to match the shape of regions to be treated (e.g., lesion, radiotherapy test area, or other areas) located at the target position. The collimation device 224 may include, for example, a multi-leaf collimator, a fiber collimator, a spherical lens collimator, etc. Optionally, the radiation generation system 22 may further include a load 227. It should be noted that in the case where the radiation delivery device 20 delivers other types of treatment radiation, the radiation generation system 22 may be adaptively configured with other arrangements or structures. For example, in the case where the radiation generation system 22 delivers electron beams, the target component and the collimation device can be omitted.

In some embodiments, the electron beam generator 221 may include an electron gun, which is configured to emit electron beams to the electron beam accelerator 222 when it is operating. It should be noted that the electron beam generator 221 is not limited to an electron gun, and for those skilled in the art, the electron beam generator 221 can be configured as other devices configured to generate electron beams, such as an electron emitter, an electron diode, etc.

In some embodiments, the electron beam accelerator 222 can be configured as a linear accelerator tube. The linear accelerator tube can specifically adopt a technical route of high-gradient and high-frequency rotation to reduce the volume required for accelerating the electron beams to high-energy electron beams required for radiotherapy during the operation of the linear accelerator tube. It should be noted that the number of linear accelerator tubes is not limited to one. It should also be understood that the electron beam accelerator 222 is not limited to a linear accelerator tube. For those skilled in the art, electron beams can also be accelerated by a spiral acceleration method, and then the high-energy electron beams from the electron beam accelerator 222 are guided into the target component 223 by a magnetic deflection component.

In addition, it should be noted that the radiation generation system 22 may also include a microwave power source 225 and a microwave transmission system 226. For example, the microwave power source 225 and the microwave transmission system 226 can deliver energy to the electron beam accelerator 222 for accelerating the electron beams.

In some embodiments, the radiotherapy apparatus 100 further includes an imaging device 40, which can be configured to acquire images of the target position (which may include the vicinity of the target position). Exemplarily, the radiation delivery device 20 can change its position based on the image acquired by the imaging device 40, so that the radiation generated by the radiation delivery device 20 is directed to the desired position. In other words, the imaging device 40 can provide image guidance for the operation of the radiation delivery device 20, thereby enabling real-time monitoring and guidance of a target region at the target position, improving the accuracy of irradiating the target object at the target position by the radiation delivery device 20 during operation, and preventing damage to normal tissues and organs around the target object. As a non-limiting example, the imaging device 40 and the radiation delivery device 20 are respectively connected to a control device 50. The control device 50 controls the position and/or orientation change of the radiation delivery device 20 based on the images acquired by the imaging device 40. However, not being limited to this manner, the radiation delivery device 20 may also change its position based on the images of the target object acquired by the imaging device 40 in other ways. For example, the imaging device 40 may be configured as a radiographic imaging device, an electronic portal imaging device (EPID), a cone-beam computed tomography device, or other imaging devices.

In some embodiments, part or all of the imaging device 40 can be positioned orthogonally to the radiation delivery device 20. The position of the imaging device 40 is adjustable relative to the treatment site 101 synchronously with the radiation delivery device 20. As such, the imaging device 40 always remains in the orthogonal position to the radiation delivery device 20, preventing the radiation generated by the radiation delivery device 20 during operation from irradiating the imaging device 40 and thereby avoiding interference with the imaging device 40. Optionally, as shown in FIG. 2, the imaging device 40 can be positioned orthogonally to the radiation delivery device 20 relative to the target position. Specifically, for example, the imaging device 40 and the radiation delivery device 20 are positioned in two perpendicular directions relative to the target position. It should be understood that in other embodiments, the imaging device 40 and the radiation delivery device 20 may be at various angles relative to the target position.

The present disclosure also provides a radiation delivery method, including delivering, by the radiation delivery device 20 movably housed within the shielding cabin 10, radiation to the target position at the treatment site 101 inside the shielding cabin 10, where the shielding cabin 10 is configured to shield radiation.

The present disclosure also provides a Non-transitory computer-readable storage medium storing instructions. The instructions, when executed by a processor, cause the processor to perform the above radiation delivery method.

In addition, the present disclosure also provides a Non-transitory computer-readable storage medium storing instructions. The instructions, when executed by a processor, implement the following steps: placing the object 200 at the treatment site 101 inside the shielding cabin 10, the shielding cabin 10 being configured to shield radiation; and delivering, by the radiation delivery device 20 movably housed within the shielding cabin 10, radiation to the object 200.

Those skilled in the art can understand that all or part of the processes in the above embodiments can be implemented by hardware instructed by a computer program. The computer program can be stored in a non-transitory computer-readable storage medium, and when executed, it can implement processes of embodiments as described above. Any memory, databases, or other media recited in the various embodiments of the present disclosure may include at least one of a non-transitory memory and a transitory memory. The non-transitory memory may include read-only memory (ROM), tape, floppy disk, flash memory, optical storage, high-density embedded non-transitory memory, resistive random-access memory (ReRAM), magnetoresistive random-access memory (MRAM), ferroelectric random-access memory (FRAM), phase change memory (PCM), graphene memory, etc. The transitory memory may include a random-access memory (RAM) or external high-speed cache memory, etc. As an illustration and not a limitation, RAM can take various forms, such as static random-access memory (SRAM) or dynamic random-access memory (DRAM), etc. The databases involved in the various embodiments of the present disclosure may include at least one of relational databases and non-relational databases. Non-relational databases may include blockchain-based distributed databases, etc., but not limited to this. The processors involved in the various embodiments of the present disclosure may be general processors, central processors, graphics processors, digital signal processors, programmable logic devices, quantum computing-based data processing logic devices, etc., but not limited to this.

In summary, according to the radiotherapy apparatus 100 of the present disclosure, a radiation solution with local self-shielding or local irradiation is employed, by which the radiation delivery device 20 is shielded with a shielding cabin 10, thereby avoiding the need to shield the entire treatment room where the radiotherapy apparatus 100 is located, which reduces costs. In addition, by adjusting the position of the radiation delivery device 20 relative to the shielding cabin 10, radiation can be directed to the target position at different angles. As such, it not only reduces the volume required to meet the demands of multi-angle irradiation of the treatment radiation, thus lowering costs, but also allows for a smaller distance between the radiation delivery device 20 and the target position, thereby further improving the therapeutic effect of the radiotherapy apparatus 100 during the radiotherapy process.

The technical features of the above embodiments can be flexibly combined. In order to keep the description concise, not all possible combinations of technical features in the above embodiments are described. However, as long as the combination of these technical features is not contradictory, it should be considered within the scope of the present disclosure.

Those skilled in the art should recognize that the above embodiments are merely intended to illustrate the present disclosure and are not intended to be limiting. Any modifications and variations made to the above embodiments within the spirit and scope of the present disclosure fall within the scope of the claims of the present disclosure.

What is claimed is:

1. A radiotherapy apparatus, comprising:
a shielding cabin, configured to shield radiation, in which a treatment site is disposed; and
a radiation delivery device, movably housed within the shielding cabin and configured to generate treatment radiation and direct the treatment radiation to a target position at the treatment site.

2. The radiotherapy apparatus according to claim 1, further comprising a guide rail arranged within the shielding cabin and configured to be movable along a length direction of the treatment site, wherein the radiation delivery device is connected to the guide rail.

3. The radiotherapy apparatus according to claim 1, further comprising a guide rail arranged around the treatment site, wherein the radiation delivery device is connected to the guide rail.

4. The radiotherapy apparatus according to claim 3, wherein the radiation delivery device is configured to be movable relative to the guide rail.

5. The radiotherapy apparatus according to claim 3, wherein the radiation delivery device is configured to be fixed relative to the guide rail and move along with the guide rail.

6. The radiotherapy apparatus according to claim 1, wherein the radiation delivery device is configured to perform a spiral motion around the treatment site.

7. The radiotherapy apparatus according to claim 2, wherein the radiation delivery device comprises a casing and a radiation generation system, and the radiation generation system is disposed within the casing and configured to generate and emit treatment radiation; and
wherein the casing is mounted to the guide rail, and the radiation generation system is configured such that an emission angle of the treatment radiation is adjustable.

8. The radiotherapy apparatus according to claim 7, wherein the radiation generation system comprises:
an electron beam generator, configured to generate electron beams;
an electron beam accelerator, configured to accelerate the electron beams derived from the electron beam generator;
a target component, configured to generate the treatment radiation using the accelerated electron beams; and
a collimation device, configured to adjust a beam shape of the treatment radiation.

9. The radiotherapy apparatus according to claim 1, wherein the shielding cabin comprises a shielding cabin chamber and a shielding cabin door, the shielding cabin door being operably mounted to the shielding cabin chamber, the shielding cabin door and the shielding cabin chamber jointly enclosing and forming an accommodating space, the treatment site being located inside the accommodating space.

10. The radiotherapy apparatus according to claim 1, further comprising an imaging device configured to acquire an image of a region of interest at the target position.

11. The radiotherapy apparatus according to claim 10, further comprising a control device communicatively connected to the imaging device and the radiation delivery device, and configured to control a position and/or an orientation of the radiation delivery device based on the image acquired by the imaging device.

12. The radiotherapy apparatus according to claim 10, wherein the imaging device is positioned orthogonally to the radiation delivery device relative to the target position.

13. The radiotherapy apparatus according to claim 12, wherein the imaging device is configured such that a position and/or orientation of the imaging device is adjustable relative to the treatment site synchronously with the radiation delivery device.

14. The radiotherapy apparatus according to claim 1, wherein the radiation delivery device is configured to be rotatable within a spherical angle range.

15. The radiotherapy apparatus according to claim 1, wherein the shielding cabin is configured to be rotated to allow for a supine or standing posture of an object.

16. A radiation delivery method based on a radiotherapy apparatus, the radiotherapy apparatus comprising a shielding cabin and a radiation delivery device movably housed within the shielding cabin, wherein the method comprises:
delivering treatment radiation, by the radiation delivery device, to a target position at a treatment site disposed in the shielding cabin, the shielding cabin being configured to shield radiation.

17. The radiation delivery method according to claim 16, wherein the radiotherapy apparatus comprises a guide rail arranged within the shielding cabin and around the treatment site, the guide rail is configured to be movable along a length direction of the treatment site, and the radiation delivery device is connected to the guide rail and configured to be movable along the guide rail.

18. The radiation delivery method according to claim 16, wherein the shielding cabin comprises a shielding cabin chamber and a shielding cabin door, the shielding cabin door being operably mounted to the shielding cabin chamber, the shielding cabin door and the shielding cabin chamber jointly enclosing and forming an accommodating space, the treatment site being located inside the accommodating space.

19. The radiation delivery method according to claim 16, wherein the radiotherapy apparatus further comprises an imaging device and a control device communicatively connected to the imaging device and the radiation delivery device, the imaging device being configured to acquire an image of a region of interest at the target position, the control device being configured to control a position and/or an orientation of the radiation delivery device based on the image acquired by the imaging device.

20. A non-transitory computer-readable storage medium storing instructions, wherein the instructions, when executed by a processor, cause the processor to perform the radiation delivery method according to claim 16.
